(19) United States
(12) Patent Application Publication
(10) Pub. No.: US 2024/0409411 A1
(43) Pub. Date: Dec. 12, 2024

(54) METHOD FOR PREPARATION OF MOF-DERIVED HIGH-POROSITY CARBON AEROGEL AS AN ELECTRODE MATERIAL OF SUPERCAPACITORS

(71) Applicant: Soochow MOFs Science and Technology Ltd., Suzhou (CN)

(72) Inventors: Zuoxi LI, Suzhou (CN); Dongyang CHEN, Suzhou (CN); Liying ZHANG, Suzhou (CN)

(21) Appl. No.: 18/811,214

(22) Filed: Aug. 21, 2024

Related U.S. Application Data
(63) Continuation of application No. PCT/CN2023/109275, filed on Jul. 26, 2023.

(30) Foreign Application Priority Data
Feb. 28, 2023 (CN) 202310174938.4

(51) Int. Cl. C01B 32/05; H01G 11/24; H01G 11/32; H01G 11/86

(52) U.S. Cl. CPC C01B 32/05; H01G 11/32; H01G 11/86; H01G 11/24

(57) ABSTRACT

The disclosure relates to the technical field of supercapacitors, in particular to a method for preparation of an MOF-derived high-porosity carbon aerogel as an electrode material of a supercapacitor. An MOFs-derived high-porosity carbon aerogel is obtained by performing calcination pyrolysis to an MOF-ZX-5 material, where the MOF-ZX-5 material is [Zn(tppa)₂Cl₂]. According to the disclosure, a new MOF-ZX-5 is used as a precursor, and a new carbon aerogel material (MOFs-derived high-porosity carbon aerogel) is designed and synthesized as an electrode material of a supercapacitor.

### CROSS-REFERENCE TO RELATED APPLICATIONS

This application is a continuation of PCT/CN2023/109275, filed Jul. 26, 2023 and claims priority of Chinese Patent Application No. 202310174938.4, filed on Feb. 28, 2023, the entire contents of which are incorporated herein by reference.

### TECHNICAL FIELD

The disclosure relates to the technical field of supercapacitors, in particular to an MOF-derived high-porosity carbon aerogel and an application thereof in supercapacitors.

### BACKGROUND

Due to the severe climate change caused by the burning of fossil fuels, the demand for clean energy such as solar energy, tidal energy and wind energy is increasing. However, these clean energy sources are greatly limited by the environment, so supercapacitors, a new energy storage system, have attracted wide attention of scientists. Supercapacitor, also known as electrochemical capacitor, is one of the most promising energy storage devices. Compared with traditional capacitors and batteries, supercapacitors have many outstanding advantages, including long repeated charge and discharge life, high power density and energy density, easy maintenance, small volume, large capacity, environmental friendliness, wide working temperature range, high high-temperature reliability and safety. Supercapacitors have a potential mechanism of reversible process at the electrode-electrolyte interface, and may provide a lasting life cycle, high power density and fast charge-discharge rate. At present, supercapacitors are widely used in consumer electronics, memory backup systems and industrial power and energy management. However, commercial carbon-based supercapacitors have low energy density, which greatly limits their use in practical applications.

Metal-organic frameworks (MOFs) are a series of new materials that form periodic multi-dimensional nanoporous materials with inorganic metal ions or ion clusters as the center and organic compounds as ligands. Compared with traditional materials, MOFs may provide rich and evenly distributed active centers, and the pore structure of MOFs is conducive to the rapid diffusion of electrolyte ions. Therefore, MOFs materials are considered as ideal supercapacitor materials. However, there is a problem in using MOFs as the anode of electrode material: after hundreds of charging and discharging cycles, the pore structure of MOFs will collapse irreversibly. This leads to a sharp decrease in the specific surface area of the electrode, which in turn leads to a decrease in the diffusion position and conductivity of electrolyte ions. This brings great challenges to the study of the conductive process of MOFs supercapacitors.

### SUMMARY

Based on the above contents, the disclosure provides an MOF-derived high-porosity carbon aerogel and an application thereof in supercapacitors. The MOF (MOF-ZX-5)-derived high-porosity carbon aerogel according to the disclosure has high specific capacitance and cycle stability as a supercapacitor material.

To achieve the above objectives, the disclosure provides following schemes.

One of the technical schemes of the disclosure is an MOF-ZX-5 material, where the MOF-ZX-5 material is [Zn(tppa)₂Cl₂]; the [Zn(tppa)₂Cl₂] is single crystal or powder crystal; crystal data of the single crystal are: monoclinic system P21/c, and an asymmetric unit includes one Zn^{II} ion, two ligand tppa molecules and two chloride ions; the [Zn(tppa)₂Cl₂] is an octahedral coordination configuration.

One of the technical schemes of the disclosure is a preparation method of the MOF material, which is a first method or a second method;
the first method includes following steps:
dissolving a ligand in a solvent, then adding a mixed solution and mixing evenly, and then adding an ethanol solution of ZnCl₂, and sealing and standing to obtain [Zn(tppa)₂Cl₂] single crystal, where the ligand is tris(4-(pyridin-4-yl)phenyl)amine;
the second method includes following steps:
dissolving a ligand in a solvent to obtain a ligand solution; adding the ligand solution dropwise into an ethanol solution of ZnCl₂, stirring, standing, performing suction filtration to obtain a precipitate, and drying the precipitate to obtain [Zn(tppa)₂Cl₂] powder crystal, where the ligand is tris(4-(pyridin-4-yl)phenyl)amine.

Further, in the first method, the solvent is chloroform (chloroform may dissolve the ligand tppa), and a molar volume ratio of the ligand to the solvent is 0.01 millimolar (mmol): 1 millilitre (mL); the mixed solution is a mixture of chloroform and ethanol with a volume ratio of 1:1 (the mixture of chloroform and ethanol has characteristic of low toxicity); a volume ratio of the solvent to the mixed solution is 3:4 (a crystal form of this product is the best under this ratio); a molar volume ratio of ZnCl₂ to ethanol in the ethanol solution of ZnCl₂ is 0.01 mmol: 3 mL; and a volume ratio of the solvent to the ethanol solution of ZnCl₂ is 1:1;

in the second method, the solvent is chloroform; a molar volume ratio of the ligand to the solvent is 0.1-0.2 mmol: 15 mL; a molar volume ratio of ZnCl₂ to ethanol in the ethanol solution of ZnCl₂ is 0.01 mmol: 3 mL; a volume ratio of the ligand solution to the ethanol solution of ZnCl₂ is 1:1; a stirring duration is 6-10 hours (h); a standing duration is 4-12 h; and a drying temperature is 50-100 degrees Celsius (° C.).

In the first method, a sealing and standing duration is 20 days, so as to cultivate a white massive single crystal structure suitable for X-ray structural analysis.

In the second method, an objective of stirring for 6-10 h and standing for 4-12 h is to synthesize powder crystals to meet the requirements of rapid industrialization, and this synthesis may not be used as structural analysis.

One of the technical schemes of the disclosure is an MOFs-derived high-porosity carbon aerogel (MOF-ZX-5-derived high-porosity carbon aerogel), which is obtained by performing calcination pyrolysis to the MOF-ZX-5 material.

One of the technical schemes of the disclosure is a preparation method of the MOFs-derived high-porosity carbon aerogel, including: performing calcination pyrolysis to the MOF-ZX-5 material to obtain the MOFs-derived high-porosity carbon aerogel.

Further, the calcination pyrolysis specifically means heating to 700-1000° C. at a rate of 3-5 degrees Celsius per minute (° C./min) under an inert atmosphere, and keeping a temperature for 2-4 h.

One of the technical schemes of the disclosure is an application of the MOFs-derived high-porosity carbon aerogel in supercapacitors.

One of the technical schemes of the disclosure is an electrode material of a supercapacitor, including the MOFs-derived high-porosity carbon aerogel.

One of the technical schemes of the disclosure is a supercapacitor, where an electrode material of the supercapacitor includes the MOF-ZX-5-derived high-porosity carbon aerogel.

The disclosure discloses following technical effects.

The gel part in MOFs-based aerogel may support the structure of MOFs to some extent, thereby improving the stability of MOFs in the cycle process. According to the disclosure, a new MOF-ZX-5 ([Zn(tppa)₂Cl₂]) is used as a precursor, and a new carbon aerogel material (MOFs-derived high-porosity carbon aerogel) is designed and synthesized as an electrode material of a supercapacitor. The MOF-ZX-5-derived high-porosity carbon aerogel has high porosity, large specific surface area and excellent capacitance performance, and is used as an electrode material of a supercapacitor with high specific capacitance, power density and energy density.

### BRIEF DESCRIPTION OF THE DRAWINGS

In order to explain the embodiments of the disclosure or the technical schemes in the prior art more clearly, the drawings needed in the embodiments will be briefly introduced below. Obviously, the drawings in the following description are only some embodiments of the disclosure, and other drawings may be obtained by ordinary people in the field without creative work.

FIG. 1 is the coordination configuration diagram of Zn^{II} ions in MOF-ZX-5 according to the disclosure.

FIG. 2 is an X-ray diffraction (XRD) spectrum of a carbon aerogel prepared in Embodiment 2.

FIG. 3 is a transmission electron microscope (TEM) image of the carbon aerogel prepared in Embodiment 2.

FIG. 4 is a Raman spectrum of the carbon aerogel prepared in Embodiment 2.

FIG. 5 shows cyclic voltammetry (CV) curves at different scan rates.

FIG. 6 shows galvanostatic charge-discharge (GCD) curves at different current densities in a three-electrode system.

FIG. 7 is a “diamond” (4,4) network structure diagram of MOF-ZX-5 according to the disclosure.

### DETAILED DESCRIPTION OF THE EMBODIMENTS

A number of exemplary embodiments of the disclosure will now be described in detail, and this detailed description should not be considered as a limitation of the disclosure, but should be understood as a more detailed description of certain aspects, characteristics and embodiments of the disclosure.

It should be understood that the terminology described in the disclosure is only for describing specific embodiments and is not used for limiting the disclosure. In addition, for the numerical range in the disclosure, it should be understood that each intermediate value between the upper limit and the lower limit of the range is also specifically disclosed. Intermediate values within any stated value or stated range, as well as each smaller range between any other stated value or intermediate values within the stated range are also included in the disclosure. The upper limit and lower limit of these smaller ranges may be independently included or excluded from the range.

Unless otherwise specified, all technical and scientific terms used herein have the same meaning as commonly understood by one of ordinary skill in the art to which this disclosure relates. Although the disclosure only describes the preferred methods and materials, any methods and materials similar or equivalent to those described herein may also be used in the practice or testing of the disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the disclosure without departing from the scope or spirit of the disclosure. Other embodiments will be apparent to the skilled person from the description of the disclosure. The description and embodiments of the disclosure are exemplary only.

The terms “comprising”, “including”, “having” and “containing” used herein are all open terms, which means including but not limited to.

“Room temperature” in the disclosure means 15-30° C. unless otherwise specified.

Unless otherwise specified, the chemicals and reagents used in the embodiments of the disclosure may be obtained from purchasing channels.

The chemicals and reagents used in the embodiments of the disclosure are commercially available analytically pure.

An electrochemical performance test method in the embodiment of the disclosure is as follows.

The electrochemical performance of the material sample (carbon aerogel) is measured by a three-electrode system at room temperature with Shanghai Chenhua Electrochemical Workstation.

Preparation of working electrode: the prepared carbon aerogel material, acetylene black and polyvinylidene fluoride are mixed according to a mass ratio of 8:1:1, and put in an agate mortar and added with a few drops of ethanol for grinding until a homogeneous black slurry is obtained, where the carbon material is an active substance, acetylene black is a conductive agent and polyvinylidene fluoride is an adhesive. Subsequently, the mixed black slurry is transferred to a pre-cleaned nickel foam with an area of 1 square centimeter (cm²) and a thickness of 2 millimeter (mm). Then, the nickel foam is put in an oven at 100° C. and dried for 12 h. Finally, the dried nickel foam is pressed by a certain high pressure (10 megapascal (MPa)) to prepare a supercapacitor electrode.

All electrochemical performance tests are carried out in a three-electrode system, and the prepared electrode is used as a working electrode, a platinum wire electrode as a counter electrode and a Hg/HgO electrode as a reference electrode. A KOH aqueous solution with a concentration of 6 mole per liter (mol/L) is used as electrolyte. Cyclic voltammetry (CV) test is carried out in a corresponding potential range (−1-0 volt (V)), with different scan rates of 5, 10, 20, 50 and 100 millivolt per second (mV s⁻¹), and curves with similar shapes and changes of current with potential are obtained.

Galvanostatic charge-discharge (GCD) test: according to the mass of the active material on the working electrode and the corresponding potential range (−1-0 V) in CV test, galvanostatic charge-discharge curves are tested at different current densities of 0.5, 1.0, 2.0, 5.0 and 10 ampere per gram (A g⁻¹). The cycle life of the carbon material is tested by galvanostatic charging and discharging at a current density of 1.0 A g⁻¹.

### Embodiment 1

### Step 1, Synthesis of [Zn(tppa)₂Cl₂], Namely MOF-ZX-5

0.4 mmol tppa is dissolved in chloroform (60 mL) to obtain a chloroform solution of tppa; 0.2 mmol ZnCl₂ is dissolved in ethanol (60 mL) to obtain an ethanol solution of ZnCl₂; the ethanol solution of ZnCl₂ is poured into a conical flask, then the chloroform solution of tppa is slowly added dropwise to the ethanol solution of ZnCl₂ through a constant pressure dropping funnel, the mixture is stirred for 6 hours at room temperature, and left to stand for 12 hours, and then suction filtration is performed to collect white precipitate, and then the white precipitate is washed with 8 mL of ethanol for 3 times. Finally, the white powder is dried at 50° C. to obtain a white powder MOF-ZX-5 crystal sample with a yield of 65%.

### Step 2, Preparation of MOF-ZX-5-Derived High-Porosity Carbon Aerogel (Abbreviated as Carbon Aerogel)

MOF-ZX-5 prepared in step 1 is put into a tube furnace, heated to 800° C. at a rate of 5° C./min under a nitrogen atmosphere, and naturally cooled to room temperature after 3 h to obtain a carbon aerogel product.

The carbon aerogel prepared in this embodiment has a specific surface area of 996 square meter per gram (m² g⁻¹) and a pore size of 2.17 nanometer (nm). As an electrode material of supercapacitors, the specific capacitance of the carbon aerogel reaches 136 farad per gram (F g 1) at a current density of 0.5 A g⁻¹, and the specific capacitance is still 133 F g⁻¹ after 2000 cycles.

### Embodiment 2

### Step 1, Synthesis of [Zn(Tppa)₂Cl₂], Namely MOF-ZX-5

0.6 mmol tppa is dissolved in chloroform (60 mL) to obtain a chloroform solution of tppa; 0.2 mmol ZnCl₂ is dissolved in ethanol (60 mL) to obtain an ethanol solution of ZnCl₂; the ethanol solution of ZnCl₂ is poured into a conical flask, then the chloroform solution of tppa is slowly added dropwise to the ethanol solution of ZnCl₂ through a constant pressure dropping funnel, the mixture is stirred for 8 hours at room temperature, and left to stand for 10 hours, and then suction filtration is performed to collect white precipitate, and then the white precipitate is washed with 10 mL of ethanol for 3 times. Finally, the white powder is dried at 80° C. to obtain a white powder MOF-ZX-5 crystal sample with a yield of 60%.

### Step 2, Preparation of MOF-ZX-5-Derived High-Porosity Carbon Aerogel (Abbreviated as Carbon Aerogel)

MOF-ZX-5 prepared in step 1 is put into a tube furnace, heated to 700° C. at a rate of 3° C./min under a nitrogen atmosphere, and naturally cooled to room temperature after 4 h to obtain a carbon aerogel product.

The carbon aerogel prepared in this embodiment has a specific surface area of 1267 m₂ g⁻¹ and a pore size of 2.51 nm. As an electrode material of supercapacitors, the specific capacitance of the carbon aerogel reaches 138 F g⁻¹ at a current density of 0.5 A g⁻¹, and the specific capacitance is still 135 F g⁻¹ after 2000 cycles.

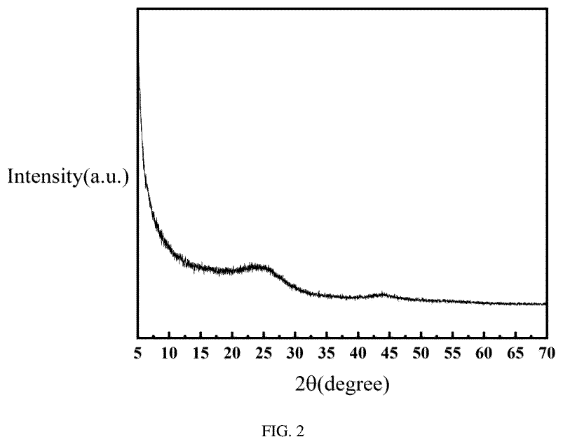

FIG. 2 is an XRD spectrum of the carbon aerogel prepared in Embodiment 2. It may be observed from FIG. 2 that the carbon aerogel has two obvious weak broad peaks around 25 and 44, which correspond to the (002) and (101) crystal planes of carbon respectively, indicating that the graphitization degree of the carbon aerogel is low.

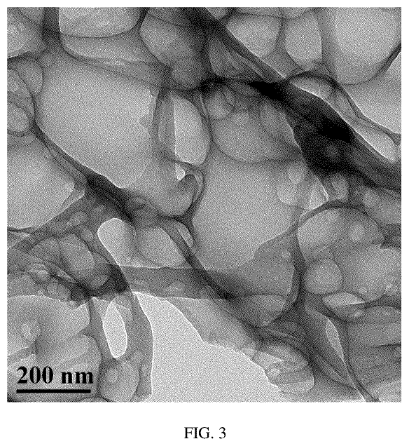

FIG. 3 is a TEM image of the carbon aerogel prepared in Embodiment 2. FIG. 2 shows the morphology and pore structure of the carbon aerogel. From FIG. 3, it may be seen that the carbon aerogel is a thin layered structure, smooth and flat, with a translucent silk shape, and a large number of disordered pore structures exist in the carbon aerogel.

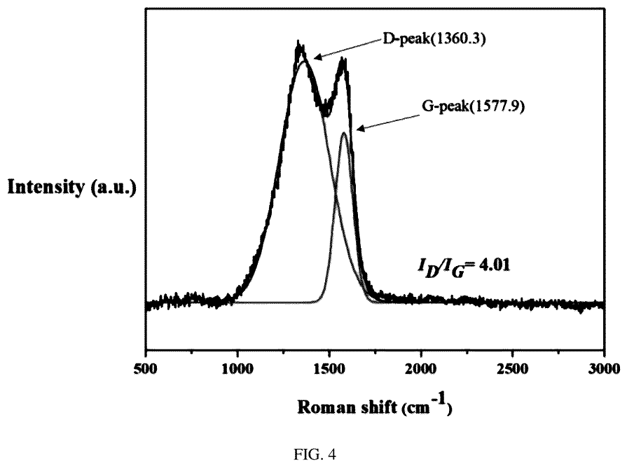

FIG. 4 is a Raman spectrum of the carbon aerogel prepared in Embodiment 2. The graphitization degree of the carbon aerogel material is analyzed by Raman spectroscopy. As shown in FIG. 4, the D peak at 1360 per centimeter (cm⁻¹) is related to the disordered carbon, and the larger the disordered defect, the stronger its strength, while the G peak at 1580 cm⁻¹ is caused by the vibration of graphitized carbon atoms. Generally, the relative ratio (I_{D}/I_{G}) of the integrated areas of D peak and G peak is used to measure the graphitization degree of carbon materials. After calculation, the I_{D}/I_{G} value of carbon aerogel prepared in Embodiment 2 is 4.01. It is speculated that the ordered structure of the carbon material of the carbon aerogel prepared may be destroyed under the conditions of this method, so that carbon atoms are randomly distributed and the graphitization degree is the smallest, thus obtaining the most fluffy product.

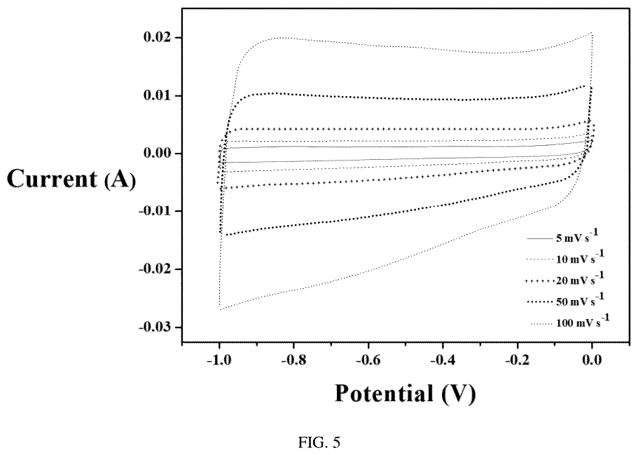

FIG. 5 shows CV curves at different scan rates. Cyclic coltammetry (CV) test is carried out in the corresponding potential range (−1-0 V) and tested at different scan rates of 5, 10, 20, 50 and 100 mV s⁻¹, and curves with similar shapes and changes of current with potential are obtained. The CV curves show a large rectangular area, and the rectangular area increases with the increase of scan rate. With the increase of scan rate and current density, the curve deformation becomes more and more serious, which is caused by the limited diffusion of electrolyte ions. For the CV curves, the specific capacitance may be calculated according to formula (1):

$$C = \int IdV / 2 v Δ Vm \qquad ( 1 )$$

where C (F g⁻¹), I (ampere) (A), V (V), v (mV s⁻¹) and m (gram) (g) respectively represent the specific capacitance, instantaneous current, voltage range, scan rate and the mass of the active material. With the increase of scan rate from 5 mV s⁻¹ to 100 mV s⁻¹, the specific capacitance of the electrode decreases from 145 F g⁻¹ to 94 F g⁻¹. This is because the electrolyte does not have enough time to reach the micropore surface at a high scan rate, resulting in less static charge stored.

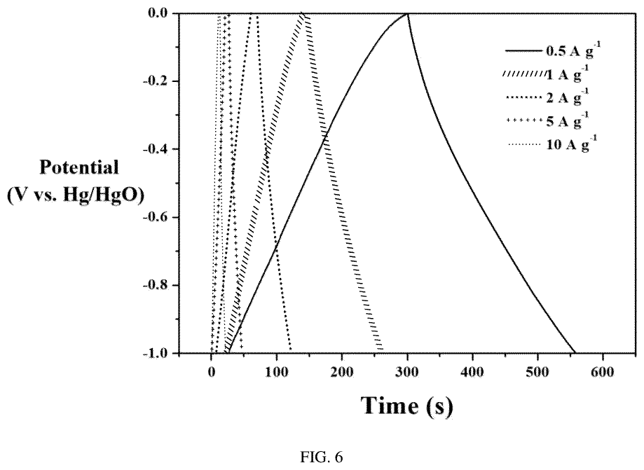

FIG. 6 shows galvanostatic charge-discharge (GCD) curves at different current densities in a three-electrode system. Under different current densities of 0.5-10 Ag⁻¹, the galvanostatic charge-discharge curves (FIG. 6) of the working electrode are tested. From the discharge curves, the specific capacitance may be obtained by formula (2).

$$C = I Δ t / Δ Vm \qquad ( 2 )$$

where t (second) (s) is the discharge time, other variables are consistent with formula (1). According to the charge-discharge test, the specific capacitance of the electrode is 130, 120, 116, 106 and 100 F g⁻¹ when the current density is 0.5, 1, 2, 5 and 10 Ag⁻¹, respectively. It is worth noting that with the gradual increase of current density, the capacitance value decreases slowly, because when the current density increases, the specific surface area that ions may reach contact decreases.

### Embodiment 3

### Step 1, Synthesis of [Zn(Tppa)₂Cl₂], Namely MOF-ZX-5

0.8 mmol tppa is dissolved in chloroform (60 mL) to obtain a chloroform solution of tppa; 0.2 mmol ZnCl₂ is dissolved in ethanol (60 mL) to obtain an ethanol solution of ZnCl₂; the ethanol solution of ZnCl₂ is poured into a conical flask, then the chloroform solution of tppa is slowly added dropwise to the ethanol solution of ZnCl₂ through a constant pressure dropping funnel, the mixture is stirred for 10 hours at room temperature, and left to stand for 8 hours, and then suction filtration is performed to collect white precipitate, and then the white precipitate is washed with 10 mL of ethanol for 3 times. Finally, the white powder is dried at 100° C. to obtain a white powder MOF-ZX-5 crystal sample with a yield of 62%.

### Step 2, Preparation of MOF-ZX-5-Derived High-Porosity Carbon Aerogel (Abbreviated as Carbon Aerogel)

MOF-ZX-5 prepared in step 1 is put into a tube furnace, heated to 1000° C. at a rate of 3° C./min under a nitrogen atmosphere, and naturally cooled to room temperature after 2 h to obtain a carbon aerogel product.

The carbon aerogel prepared in this embodiment has a specific surface area of 1293 m² g⁻¹ and a pore size of 4.24 nm. As an electrode material for supercapacitors, the specific capacitance of the carbon aerogel reaches 142 F g⁻¹ at a current density of 0.5 Ag⁻¹, and the specific capacitance is still 137 F g⁻¹ after 2000 cycles.

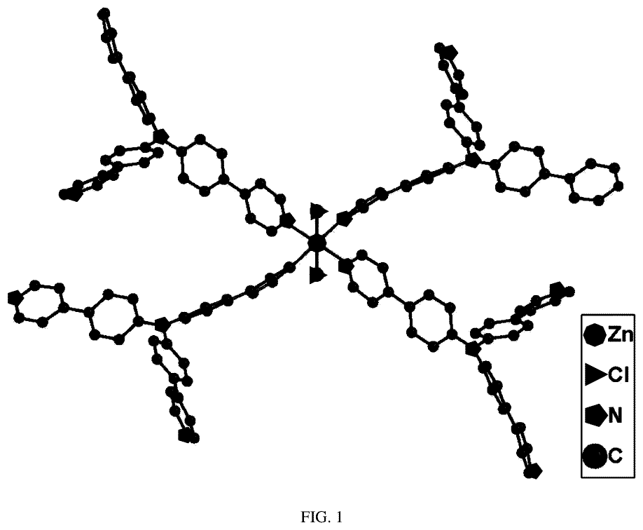

FIG. 1 is a coordination configuration diagram of Zn^{II} ions in MOF-ZX-5 according to the disclosure. FIG. 1 shows the structural analysis of MOF-ZX-5 single crystal. A space group of MOF-ZX-5 belongs to monoclinic system P2_{1/c}, and an asymmetric unit includes one Zn^{II} ion, two ligand tppa molecules and two chloride ions. As shown in FIG. 1, each Zn^{II} ion coordinates with four nitrogen atoms from different tppa molecules and two chloride ions to form an octahedral coordination configuration. The length of the Zn—N coordination bond in the equatorial plane is between 2.167-2.186 angstrom (Å), while the length of the Zn—Cl coordination bond in the axial direction is 2.486 Å. Therefore, the Jahn-Teller effect obviously exists in Zn^{II} ions.

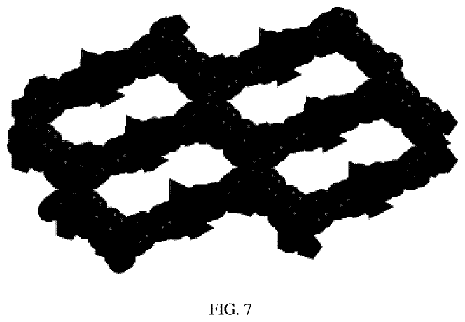

FIG. 7 is a “diamond” (4,4) network structure diagram of MOF-ZX-5 according to the disclosure. Although the tppa molecule has three nitrogen atoms, only two nitrogen atoms participate in the coordination during the assembly process, that is, each tppa ligand bridges two Zn^{II} ions to obtain a two-dimensional “diamond” (4,4) network with a lattice size of 12.2×12.2 Å² (FIG. 7), and the pore size reaches the nanometer level.

Carbon-based electrode materials used in supercapacitors have good reversibility, fast charging ability, long cycle life and good environmental friendliness, but the relatively low specific capacitance leads to low energy density, which restricts the comprehensive application. As a classical porous compound, MOFs has good structural characteristics such as long-range ordered structure and large specific surface area. According to the disclosure, a new macroporous MOF-ZX-5 is used as a precursor to synthesize a new carbon aerogel electrode material, so that the specific capacitance and energy density of the electrode material of the supercapacitor are improved.

The above-mentioned embodiments only describe the preferred modes of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. A method for preparation of MOF (MOF-ZX-5)-derived high-porosity carbon aerogel as an electrode material of a supercapacitor, comprising the following steps:
dissolving a ligand in a solvent to obtain a ligand solution; adding the ligand solution dropwise into an ethanol solution of ZnCl₂, stirring, standing, performing suction filtration to obtain a precipitate, and drying the precipitate to obtain the [Zn(tppa)₂Cl_{2]},
wherein the ligand is tris(4-(pyridin-4-yl)phenyl)amine, and the MOFs-derived high-porosity carbon aerogel is obtained by performing calcination pyrolysis to an MOF-ZX-5 material by heating to 700-1000° C. at a rate of 3-5° C./min under an inert atmosphere, and keeping at the temperature for 2-4 h.

2. The method of claim 1, wherein the MOF-ZX-5 material [Zn(tppa)₂Cl₂] is single crystal or powder crystal; crystal data of the single crystal are: monoclinic system P21/c, and an asymmetric unit comprises one Zn^{II} ion, two ligand tppa molecules, and two chloride ions; the [Zn(tppa)₂Cl₂] is an octahedral coordination configuration.

3. The method of claim 1, wherein the solvent is chloroform; a molar volume ratio of the ligand to the solvent is 0.1-0.2 mmol: 15 mL; a molar volume ratio of ZnCl₂ to ethanol in the ethanol solution of ZnCl₂ is 0.01 mmol: 3 mL; a volume ratio of the ligand solution to the ethanol solution of ZnCl₂ is 1:1; a stirring duration is 6-10 h; a standing duration is 4-12 h; and a drying temperature is 50-100° C.
